(19) United States
(12) Patent Application Publication
(10) Pub. No.: US 2005/0077436 A1
(43) Pub. Date: Apr. 14, 2005

(54) Organizer for medical tubes and cables

(76) Inventor: Deborah K. Nelson, Amarillo, TX (US)

Correspondence Address: BRACEWELL & PATTERSON, L.L.P., P.O. BOX 61389, HOUSTON, TX, 77208-1389, US

(21) Appl. No.: 10/961,287

(22) Filed: Oct. 8, 2004

Related U.S. Application Data
(63) Continuation-in-part of application No. 10/323,270, filed on Dec. 20, 2002, now abandoned.

(51) Int. Cl. F16L 3/22; A61M 5/14

(52) U.S. Cl. 248/68.1; 248/72

(57) ABSTRACT

A device for organizing medical lines so as to facilitate identification and prevent entanglement and dislodgement of the lines includes a rigid elongated base panel and an upwardly directed securing panel. Mounting features located beneath the base panel enable the apparatus to be quickly and removably attached to virtually any type of rail. A series of slots in the securing panel extend toward the base panel and terminate in confining apertures having dimensions larger than the width of the associated slot. The device permits transverse insertion of a medical line into a slot and positioning in a confining aperture, thereby allowing longitudinal and limited lateral movement of the line while preventing entanglement with adjacent lines.

The present patent application is a continuation-in-part of U.S. patent application No. 10/323,270, filed on Dec. 20, 2002, entitled, Organizer for Medical Tubes and Cables, and is incorporated herein by reference.

### BACKGROUND OF THE INVENTION

1. Technical Field

This invention relates to an apparatus for neatly organizing medical lines that administer to a patient and, more particularly, to an apparatus that functionally secures a plurality of flexible cables and tubes interactive with a patient and, even more particularly, to the management and identification of multiple IV lines adjacent to a patient.

2. Description of the Related Art

The subject matter of this invention will be discussed primarily in conjunction with flexible lines in the form of fluid-conducting tubes that are commonly used within the medical profession. However, it is considered to be within the scope of this invention that the apparatus of the invention could be utilized in conjunction with lines other than tubes such as, for example, electrical and fiber optic cables employed for monitoring or diagnostic purposes.

During the course of medical procedures, a human patient is often infused simultaneously with a plurality of different medications and fluids. Those medications and fluids are fed into the patient through tubes. Additionally, catheters are frequently utilized to monitor body functions. Many of these catheter, fluid-conducting tubes and monitoring lines are placed on the patient prior to when the patient is transported from a surgery area to a convalescing area. These different lines have a tendency to become entangled, making it difficult for attending physicians and nurses to ascertain which line is being utilized for which purpose. At times certain medications are injected into a fluid line. Extreme care has to be exercised to make sure that the right medicine is being injected into the correct line. This problem of confusing lines and catheters entering a patient's body is accentuated under emergency conditions. The foregoing problems increase the risk of patient injury by either infusing medicine or blood at the wrong entry site, or by failure to infuse needed medications.

The entanglement of the different lines is exceedingly common in the hospital room while the patient is recovering from a surgical operation. The entanglement is caused primarily by the patient moving around in either a conscious or unconscious condition. Some means is therefore needed in a hospital room for organizing the various medical lines servicing a bedridden patient, said organization facilitating the location and identification of each line and preventing their entanglement and dislodgement.

Devices for organizing medical lines that service a patient in a hospital bed are disclosed in U.S. Pat. Nos. 5,334,186; 5,336,179; 5,427,338; 5,624,403; 5,876,371 and elsewhere.

Most earlier organizer devices restrict movement of the lines they secure. Such restriction enables tensile stress to be placed upon a line with movement of the patient. The resultant stress can cause the medical line to separate from its interaction with the patient or with a monitoring instrument. Earlier medical line organizers may also be incompatible with typical hospital beds or may be difficult to attach thereto. The individual lines may not be easily engageable by the organizer, and inadequate means are provided for the identification of each line.

It is accordingly an object of the present invention to provide apparatus for organizing medical lines interactive with a patient.

It is another object of this invention to provide an organizer apparatus as in the foregoing object adapted to separately constrain a number of medical lines without preventing axial or lateral motion thereof.

It is a further object of the present invention to provide an organizer apparatus of the aforesaid nature, which is easily securable to a hospital bed or other structures, and readily accepts said medical lines.

It is yet another object of this invention to provide an organizer apparatus of the aforesaid nature that readily permits association of identifying indicia with each of said medical lines.

It is a still further object of the present invention to provide an organizer apparatus of the aforesaid nature of durable, compact construction amenable to sterilization treatment.

It is an additional object of this invention to provide an organizer apparatus of simple construction amenable to sufficiently low cost manufacture as to justify disposal following limited use.

These objects and other objects and advantages of the invention will be apparent from the following description.

### SUMMARY OF THE INVENTION

The above and other beneficial objects and advantages are accomplished in accordance with the present invention by an organizer apparatus for mounting upon a rail for receiving flexible medical lines. One embodiment of the present invention comprises:
a) a rigid base panel of elongated rectangular contour bounded in part by a upper surface and opposed inner surface,
b) mounting means associated with said inner surface for the purpose of releasably engaging a rail, and
c) a securing panel orthogonally emergent from said upper surface and terminating in a distal extremity parallel to said upper surface, and having a series of spaced apart slots which open upon said distal extremity and terminate in a confining aperture within said securing panel and dimensioned larger than the width of the associated slot, whereby
d) flexible medical line can be laterally inserted through a slot and caused to reside within an associated confining aperture which permits axial and lateral sliding movement of the line, and said upper surface permits placement of indicia to identify each confined line.

### BRIEF DESCRIPTION OF THE DRAWINGS

For a fuller understanding of the nature and objects of the invention, reference should be had to the following detailed description taken in connection with the accompanying drawing forming a part of this specification and in which similar numerals of reference indicate corresponding parts in all the figures of the drawing.

FIG. 1 is a top and side perspective view of one embodiment of the organizer apparatus of the present invention.

FIG. 2 is a top view of the embodiment of FIG. 1.

FIG. 3 is a side view of the embodiment of FIG. 1.

FIG. 4 is an end view of the embodiment of FIG. 1.

FIG. 5 is an isometric view of the embodiment of FIG. 1 secured to a bed rail.

FIG. 6 is an isometric view of the embodiment of FIG. 1 secured to a vertically-oriented pole.

### DETAILED DESCRIPTION OF THE INVENTION

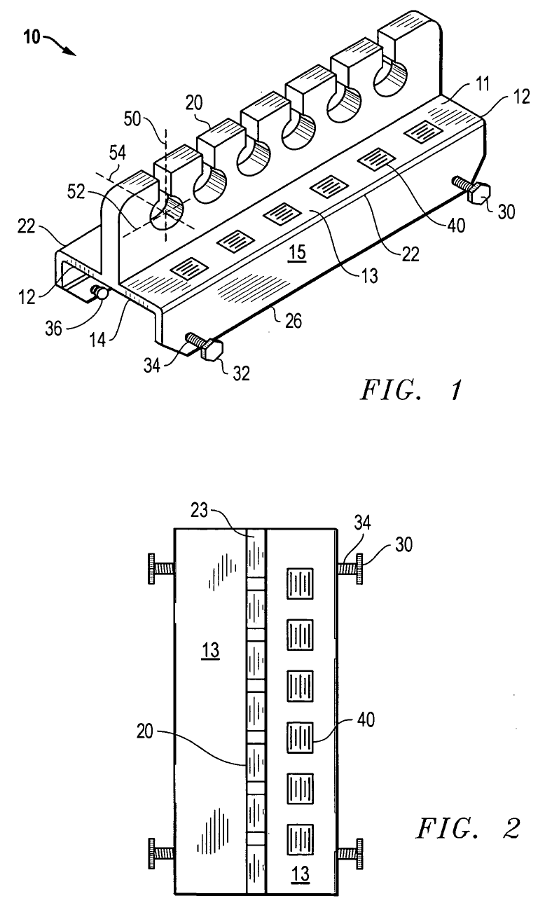

Referring now to FIGS. 1-6, one embodiment of the organizer apparatus 10 of the present invention is shown as a monolithic structure which may be fabricated of thermoplastic material by way of a molding operation or by an extrusion operation with a subsequent punching or machining step. This design facilitates the disposability of the present invention following limited use. However, the unit also can be cold sterilized for re-use if so desired.

The organizer apparatus is shown comprised of a rigid base panel 11 of generally rectangular contour elongated between end extremities 12 and bounded in part by flat upper or outer surface 13 and an opposed lower or inner surface 14. The length of said base panel, measured between said end extremities may range from about 6 to 12 inches. The width of upper surface 13, measured between opposed side edges 22 may range from about 1 to 3 inches.

Opposed side panels 15, downwardly directed from said lower surface, are spaced apart a proper distance to frictionally embrace a bed rail. Said side panels, in concert with said lower surface thereby function as mounting means that permit rapid, releasable attachment of the apparatus to a bed rail. A layer of rubbery material 17 may be adhered to the facing surfaces 18 of said side panels for the purpose of preventing slippage of the apparatus while mounted upon the bed rail. Said side panels may typically have a height of 1 to 3 inches, as measured between upper surface 13 and the lower edge 26 of said side panel.

In the embodiment exemplified in FIGS. 1-6, side panels 15 are downwardly convergent. Such configuration enables the side panels to operate resiliently to embrace the bed rail. However, the side panels 15 may extend orthogonally from the base panel 11 such that they are parallel to each other. The diameter of most hospital bed rails is 1.378 inches. By causing the lowermost edges 26 of said side panels to be spaced slightly less than 1.378 inches apart, the side panels function as a spring-like mounting clip which receives and secures a rail.

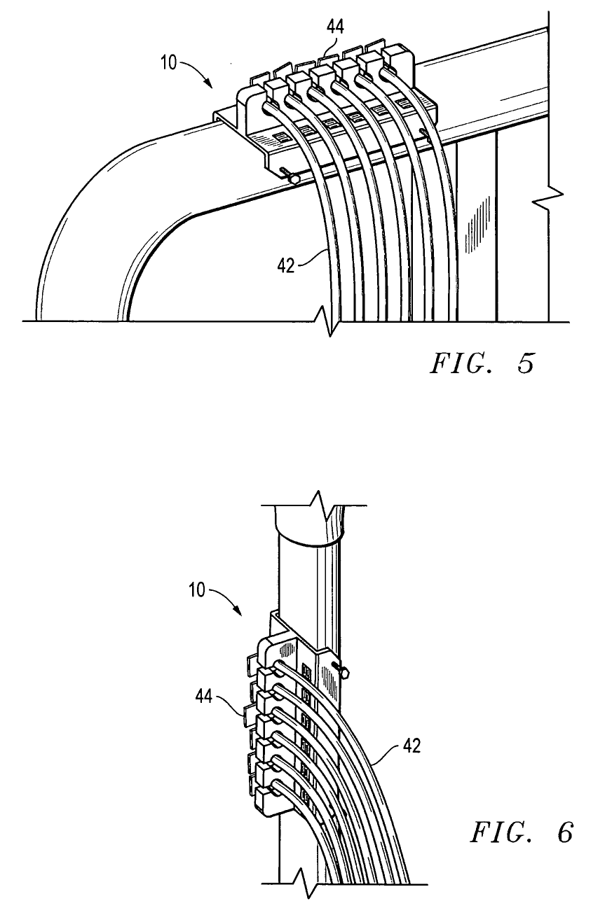

In addition, at least one of the side panels 15 is provided with at least one retention device 30. In the embodiment shown, each side panel 15 has two retention devices 30, which are thumbscrews. The retention devices 30 are adjustable in length along their axes with respect to side panels 15 in order to accommodate a wide range of sizes, types (e.g., round, square, etc.), and orientations (e.g., horizontal, vertical, etc.) of rails (see FIGS. 5 and 6) to which apparatus 10 may be attached. The retention devices 30 may be extended through the rubbery material 17 to rigidly secure the apparatus 10 thereto and effectively prevent motion therebetween.

The retention devices 30 may be formed from the same material as the remainder of the apparatus 10. In addition, the retention devices 30 may be identical to each other or provided in different sizes, depending on the application. In the version illustrated, each retention device 30 is provided with a head 32, a threaded shaft 34 (which engages a tapped hole in side panel 15), and a flanged end 36 opposite head 32. The head 32 may be provided with a hexagonal shape and/or knurled about its perimeter and is sized so that it may be grasped and turned easily by a user. The flanged end 36 ensures that the retention device 30 is not easily removed or lost from the side panel. The retention devices 30 on each side panel 15 are centered with respect to axis 50 on side panel 15. In addition, each retention device 30 is offset from their respective edges 12 by about one-fourth of the overall length of base panel 11.

A securing panel 20 is, in the embodiment shown, orthogonally emergent from upper 10 surface 13 in parallel relationship to side edges 22. Said securing panel is preferably centered upon upper surface 13, thereby causing the apparatus to have a longitudinal plane of symmetry 21. Securing panel 20 is upwardly terminated by straight distal extremity surface 23 parallel to upper surface 13. Said distal surface has a series of spaced apart slots 24 elongated toward said upper surface and terminating in a confining aperture 25 within said securing panel. Slots 24 preferably have a widened upper extremity in the form of an upwardly flared configuration 27 which facilitates insertion of a line 42 laterally (along axis 50 in FIG. 1), namely in a direction transverse to the direction of elongation of said line. Slots 24 can be straight or tapered. In one version, slots have a width between about 3 and 10 millimeters at their narrowest point, and can be varied within the organizer apparatus.

A confining aperture 25, as measured in both horizontal and vertical directions relative to upper surface 13, is larger than the narrowest width of associated slot 24. Confining apertures 25 may typically have a circular, oval or rectangular configuration and is much larger in diameter, or major/minor axes, than the diameter of the lines 42 that they secure. It is important that said configuration be horizontally elongated, namely elongated upon axis 52, parallel to upper surface 13. The apertures 25 also have a thickness along axis 54 that, in the embodiment shown, is the same as the thickness of panel 20. In some embodiments, securing panel 20 may be fabricated of a resilient or rubbery material, and attached to base panel 11.

By virtue of the aforesaid construction, the organizer apparatus 10 (FIG. 1) can accommodate medical lines 42 (shown with clips or valves 44) in an orderly manner. The medical lines may be tubes that convey liquids or gasses to or from the patient, or may be electrical or fiber optic cables utilized to monitor or otherwise administer to the patient's condition or needs. Such medical lines, when laterally inserted through a slot, are caused to reside within an associated confining aperture in a manner that permits axial and lateral sliding movement of the lines. Thus, although the lines 42 are retained in apparatus 10, they are free to move back and forth along axis 54, and even have a limited range of motion along axes 50 and 52. The flat upper surface 13 permits placement of indicia 40 to identify such confined line. Suitable indicia 40 may include, for example, self-adhesive labels, pen markings, etc. The indicia 40 may also include instructional information. On/off control valves, clamps, hypodermics or other operational features may be associated with appropriate lines adjacent the organizer apparatus.

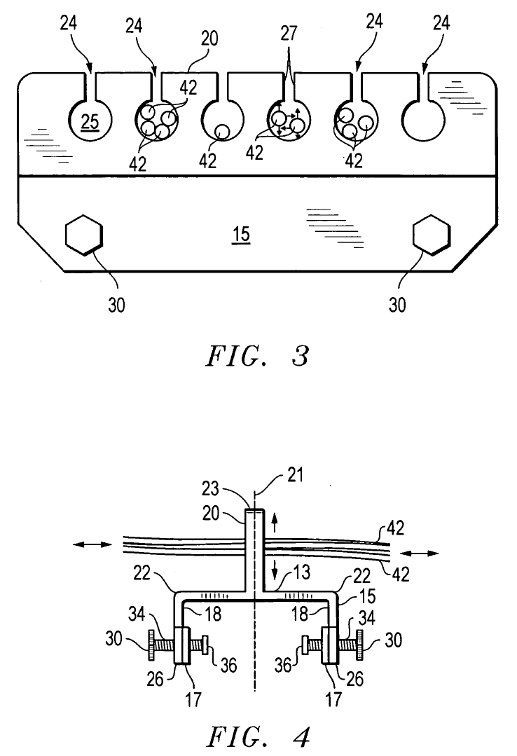

Referring once again to FIGS. 3 and 4, in one embodiment, multiple ones of the lines 42 may be positioned in at least one of the apertures 25 as shown. This design is particularly well suited for critical care patients. Even with more than one line 42 in one of the apertures 25, the aperture 25 is sized to provide ample room for the multiple lines 42 to simultaneously move both axially (e.g., longitudinally along axis 54 in FIG. 1) and laterally (e.g., vertically and horizontally along axes 50, 52). This ample provision of mobility for the lines 42 is important to prevent the lines 42 (i.e., and their catheters from the patient's veins, etc.) from being dislodged or removed from the patient to whom they are attached, particularly when the patient rolls over or moves suddenly or quickly. Even small amounts of friction between the line(s) 42 and their respective aperture 25 (such as that disclosed in U.S. Pat. No. 6,458,104) can facilitate the accidental removal of the lines 42 from the patient's body. Thus, as shown in FIG. 3, the diameter of aperture 25 is much larger than the diameter of a line 42, and is also significantly larger than the combined diameters of multiple ones of the lines 42.

Moreover, in one embodiment, the size of the opening (e.g., the “width opening”) in at least one of the slots 24 (e.g., in the direction of axis 52) is smaller than the diameter of one of the lines 42, as shown. Ideally, the width openings of the slots 24 are smaller than a diameter of the smallest line 42. Thus, a line 42 will not simply “slip out” of slot 24 without being forcibly squeezed through it by an outward pull (e.g., away from panel 20 in the direction of axis 50) through slot 24. Even a slight about of flexibility in the sidewalls of a line 42 permit this geometric configuration. For example, a line 42 may be grasped in two places (i.e., on both sides of panel 20), and then pulled outward from aperture 25 and slot 24. As the line 42 moves from aperture 25 through slot 24, its sidewalls undergo a slight elastic deformation to permit its removal from panel 20. This size restriction of slot 24 prevents the accidental removal of one or more of the lines 42 from aperture 25 without the need of a clamp or closure. See, clamp 70 in FIG. 3 of U.S. Pat. No. 6,458,104. The lack of a clamp or closure also permits an attendant to move or replace individual lines 42 without exposing the entire set of lines 42 to the risk of imminent dislodgement by a patient.

The present invention has many other advantages as well. For example, the use of patient safety devices and techniques are emphasized by governmental initiatives in hospitals (e.g., The Joint Commission on Hospital Accreditation). The present invention is the only IV organizer design that meets the requirements of these initiatives. More specifically, it both contains features that prevent IV medication errors (e.g., placing medication in the wrong line) and interruption of said medication administration by means of a patient accidentally dislodging one or more of his or her IV lines. The most critical patients, which are most likely to need multiple intravenous medications, are also the most often sedated, agitated or obtunded, and therefore also the most likely to disengage themselves from life-supporting medications without knowing anything has gone wrong. IV pumps have alarms to signify that a line has been pinched off, but none to indicate that the medication is flowing freely onto the patient's bed sheets. The present invention is a failsafe in this regard. Anyone in the federal healthcare bureaucracy would immediately identify this design as a unique and needed addition to the list of technologies geared toward preventing medication mishaps.

While particular examples of the present invention have been shown and described, it is apparent that changes and modifications may be made therein without departing from the invention in its broadest aspects.

1. An organizer apparatus, comprising:
a base panel;
mounting means on the base panel and having a retention device for releasably retaining the base panel on a rail;
a securing panel extending from the base panel, the securing panel having a terminal surface; and
a series of spaced-apart slots formed in the securing panel and extending from an interior of the securing panel to the terminal surface, the slots defining width openings for permitting entry of a line, and apertures that are dimensioned larger than the width openings for retaining the line, such that multiple lines can be laterally inserted through at least one of the slots and caused to reside within an associated one of the apertures that permits simultaneous axial and lateral sliding movement of the multiple lines.

2. The organizer apparatus of claim 1, wherein the width opening of said at least one of the slots has a dimension that is smaller than a diameter of the lines, and said associated one of the apertures defines a dimension that is greater than the diameter of the lines.

3. The organizer apparatus of claim 2, wherein the dimension of said associated one of the apertures is greater than a combination of the diameters of the multiple lines, such that said one of the apertures permits simultaneous axial and lateral sliding movement of the multiple lines.

4. The organizer apparatus of claim 1, wherein said mounting means comprises a pair of opposed side panels having facing surfaces that are spaced apart from each other, the side panels acting in concert with the base panel to retain the organizer apparatus on the rail.

5. The organizer apparatus of claim 4, wherein each side panel has a layer of rubbery material attached to a facing surface for preventing slippage of the organizer apparatus while mounted on the rail.

6. The organizer apparatus of claim 4, wherein the two side panels converge toward each other and terminate with lowermost parallel edges that are spaced apart from each other by a distance that is less than a diameter of the rail.

7. The organizer apparatus of claim 1, wherein each slot has a widened upper extremity that opens upon the terminal surface and facilitates transverse insertion of the line.

8. The organizer apparatus of claim 1, wherein the retention device comprises a thumbscrew.

9. The organizer apparatus of claim 4, wherein the retention device comprises a plurality of thumbscrews mounted to and extending through the side panels, each of the thumbscrews having an axis and being axially movable relative to the side panels to secure the organizer apparatus to the rail.

10. A system of organizing lines on a rail, the system comprising:
a base panel mounted to the rail, the base panel having an outer surface and opposed inner surface in contact with the rail;
a securing panel extending from the outer surface and having a terminal surface that is a distal-most portion of the securing panel relative to the outer surface, the securing panel having an elongated width in a direction that is parallel to the outer surface;
a series of spaced-apart slots formed in the securing panel and extending from an interior of the securing panel to the terminal surface, apertures extending from the slots that are dimensioned larger than a width of an associated one of the slots, such that multiple lines are laterally inserted through one of the slots and caused to reside within an associated one of the apertures that permits axial and lateral sliding movement of the lines therein, and the width of said associated one of the slots is less than a diameter of one of the lines; and
retention means for securing the base panel to the rail.

11. The system of claim 10, wherein the dimensions of the apertures is greater than a combination of the diameters of the multiple lines, such that the apertures permit simultaneous axial and lateral sliding movement of the multiple lines.

12. The system of claim 10, wherein the base panel is mounted to the rail with a pair of opposed side panels having facing surfaces that are spaced apart from each other, the side panels acting in concert with the inner surface of the base panel for retaining the base panel on the rail.

13. The system of claim 12, wherein the retention device comprises a plurality of thumbscrews mounted to and extending through the side panels, each of the thumbscrews having an axis and being axially movable relative to the side panels to secure the organizer apparatus to the rail.

14. The system of claim 12, wherein:
each side panel has a layer of rubbery material attached to its facing surface for preventing slippage of the system with respect to the rail; and
the two side panels converge toward each other and terminate with lowermost parallel edges that are spaced apart from each other by a distance that is less than a diameter of the rail.

15. The system of claim 10, wherein each slot has a widened upper extremity that opens upon the terminal surface for transverse insertion of the flexible lines.

16. A method of organizing medical lines, comprising:
(a) providing an organizer with a panel having a plurality of slots formed therein, each slot terminating in an aperture;
(b) mounting the organizer on a rail;
(c) inserting a plurality of medical lines through a width opening in one of the slots such that the plurality of medical lines reside in a respective aperture associated with said one of the slots; and then
(d) moving the plurality of medical lines both axially and laterally with respect to said respective aperture.

17. The method of claim 16, further comprising dimensioning a diameter of said respective aperture larger than the width opening of said one of the slots, and dimensioning the width opening of said one of the slots smaller than a diameter of one of the medical lines.

18. The method of claim 17, further comprising dimensioning the diameter of said respective aperture larger than a combination of the diameters of the multiple lines, such that said respective aperture permits simultaneous axial and lateral sliding movement of the multiple lines, and dimensioning the width opening of said one of the slots smaller than a diameter of a smallest one of the medical lines.

19. The method of claim 16, wherein step (b) comprises tightening at least one thumbscrew on the organizer to engage the rail.
